(19) United States
(12) Patent Application Publication
(10) Pub. No.: US 2013/0275053 A1
(43) Pub. Date: Oct. 17, 2013

(54) COMPUTER IMPLEMENTED METHOD FOR ANALYZING A GAS SAMPLE USING AN INLINE GAS ANALYZER

(71) Applicant: SELMAN AND ASSOCIATES, LTD., Midland, TX (US)

(72) Inventors: Thomas H. Selman, Midland, TX (US); Matthew J. Jennings, Midland, TX (US); Stephen M. Bergman, Casper, WY (US)

(73) Assignee: SELMAN AND ASSOCIATES, LTD., Midland, TX (US)

(21) Appl. No.: 13/863,970

(22) Filed: Apr. 16, 2013

Related U.S. Application Data
(60) Provisional application No. 61/625,357, filed on Apr. 17, 2012.

(51) Int. Cl. G01N 33/00

(52) U.S. Cl. CPC G01N 33/0004
USPC 702/24

(57) ABSTRACT

A computer implemented, real-time, continuous, electromagnetic, spectroscopic, high sensitivity, digital method for analyzing a gas sample can include using an inline gas component analyzer to receive the gas sample. An electromagnetic beam generator can generate a beam to pass through a filter in a sample chamber and into the sample gas to form a sample wavelength. The sample wavelength can pass into an electromagnetic beam detector. A processor and data storage can be in communication with each component of the inline gas component analyzer for monitoring and controlling thereof. The processor can be in communication with client devices through a network for remote monitoring and controlling thereof. The inline gas component analyzer can be calibrated with a calibration gas sample.

### CROSS REFERENCE TO RELATED APPLICATION

The present application claims priority to and the benefit of co-pending U.S. Provisional Patent Application Ser. No. 61/625,357 filed on Apr. 17, 2012, entitled “COMPUTER IMPLEMENTED METHOD FOR ANALYZING A GAS SAMPLE USING AN INLINE GAS ANALYZER.” This reference is hereby incorporated in its entirety.

### FIELD

The present embodiments generally relate to a computer implemented method for analyzing a gas sample using an inline gas analyzer.

### BACKGROUND

A need exists for a method for a computer implemented real time continuous electromagnetic spectroscopic high sensitivity digital method for analyzing a gas sample using an inline chromatograph and inline gas totalizer.

A need exists for a method that can provide scanned pictures of a gas sample.

A need exists for a method that can transmits a beam through the filter into the gas sample very quickly, such as at 1000 pulses per minute.

A need exists for a method that can analyze a sample gas from a drilling fluid by providing a continuous wavelength sweep of a single wavelength band or a continuous sweep of multiple wavelength bands simultaneously through the gas sample.

A need exists for a method that can analyze gas that is not always at a perpendicular angle using a filter that can be tilted away from a position perpendicular to the electromagnetic beam.

A need exists for a method that can use minors to allow the electromagnetic beam to go through the filter and bounce back to an electromagnetic detector creating a wavelength sweep of the gas, hitting different points of the gas.

The present embodiments meet these needs.

### BRIEF DESCRIPTION OF THE DRAWINGS

The detailed description will be better understood in conjunction with the accompanying drawings as follows:

FIG. 1A depicts an embodiment of a system that can be used to implement the method for testing a gas sample.

FIG. 1B depicts the system of FIG. 1A testing a calibration gas sample.

FIG. 2 depicts an embodiment of the system having two sample chambers.

FIG. 3A depicts an embodiment of the filter frame having a single filter.

FIG. 3B depicts an embodiment of the filter frame having a plurality of filters.

FIG. 4 depicts the transmission of the beam through filters.

FIG. 5A depicts an embodiment of a calibration radiation matrix.

FIG. 5B depicts an embodiment of an overall spectral data matrix.

FIG. 5C depicts a response curve from a single component of a gas sample.

FIGS. 6A and 6B depict a data storage having a plurality of computer instructions for implementing the method according to one or more embodiments.

FIGS. 7A, 7B, and 7C depict an embodiment of the method.

The present embodiments are detailed below with reference to the listed Figures.

### DETAILED DESCRIPTION OF THE EMBODIMENTS

Before explaining the present method in detail, it is to be understood that the method is not limited to the particular embodiments and that it can be practiced or carried out in various ways.

The present embodiments relate to a method for analyzing a gas sample using an inline gas analyzer.

The method can be computer implemented, including use of various computers, communication networks, computer readable medium, and computer instructions to perform or assist in performing one or more portions of the method.

The method can provide for digital analysis of the gas samples by providing analyzed results via computers and networks as a feed of digital data, including a continuously streaming feed of analyzed values.

The method can provide for real-time analysis, such that the method can provide a real-time frequency of detection, a real-time frequency of analysis results, or combinations thereof.

The method can provide for continuous analysis by: analyzing a constant sample flow entering a sample chamber, constantly detecting samples, constantly analyzing the detected samples, and constantly providing analysis results of the detected samples.

The method can provide for electromagnetic analysis of gas samples by using electromagnetic radiation to ionize components of gas samples, illuminate components of gas samples, allow absorption of spectra by components of gas samples, or combinations thereof.

The method can provide for spectroscopic analysis of gas samples by analyzing responses from gas samples over a range of wavelengths.

The method can provide for high sensitivity analysis of gas samples by: providing quick responses for detection of gas samples, such as detecting the gas samples within 2 seconds after introduction of the gas samples into an inline component gas analyzer; and providing accurate detection of the gas samples, such as providing measurements having values within a 5 percent tolerance of measured values.

The method can provide for extremely high sensitivity analysis of gas samples by providing measurements having values having tolerances from 1 percent to 2 percent of measured values.

The inline component gas analyzer can include a gas chromatograph and gas totalizer combined assembly; a spectroscopic elemental analyzer using multiple frequencies with multiple filters; a spectroscopic elemental analyzer using multiple frequencies with multiple filters and gas totalizer combined assembly; or combinations thereof.

The inline component gas analyzer can include infrared detectors, near infrared detectors, flame ionization detectors (FID), thermal conductivity detectors (TCD), catalytic combustion detectors (CCD), photoionization detectors, electromagnetic spectrometers, or combinations thereof.

In operation, the method can provide scanned pictures of the gas samples as the gas sample moves across a filter by transmitting a beam through the filter and into the gas sample, such as at a rate of about 1000 pulses of the beam per minute. The beam can be an electromagnetic beam.

The method can include analyzing gas samples from a drilling fluid from a well.

The method can include continuously sweeping the gas sample with the beam to provide a continuous sweep at a single wavelength or a continuous sweep at multiple wavelengths simultaneously.

In operation, the gas sample can be disposed at a perpendicular angle to the beam or at an angle that is not perpendicular to the beam by using a filter that can be tilted away from a position perpendicular to the beam.

The method can include using mirrors to allow the beam to pass through the filter, into the gas sample, bounce back through the gas sample and the filter, and be reflected to an electromagnetic beam detector; thereby creating a wavelength sweep of the gas sample by transmitting the beam through the gas sample at different points.

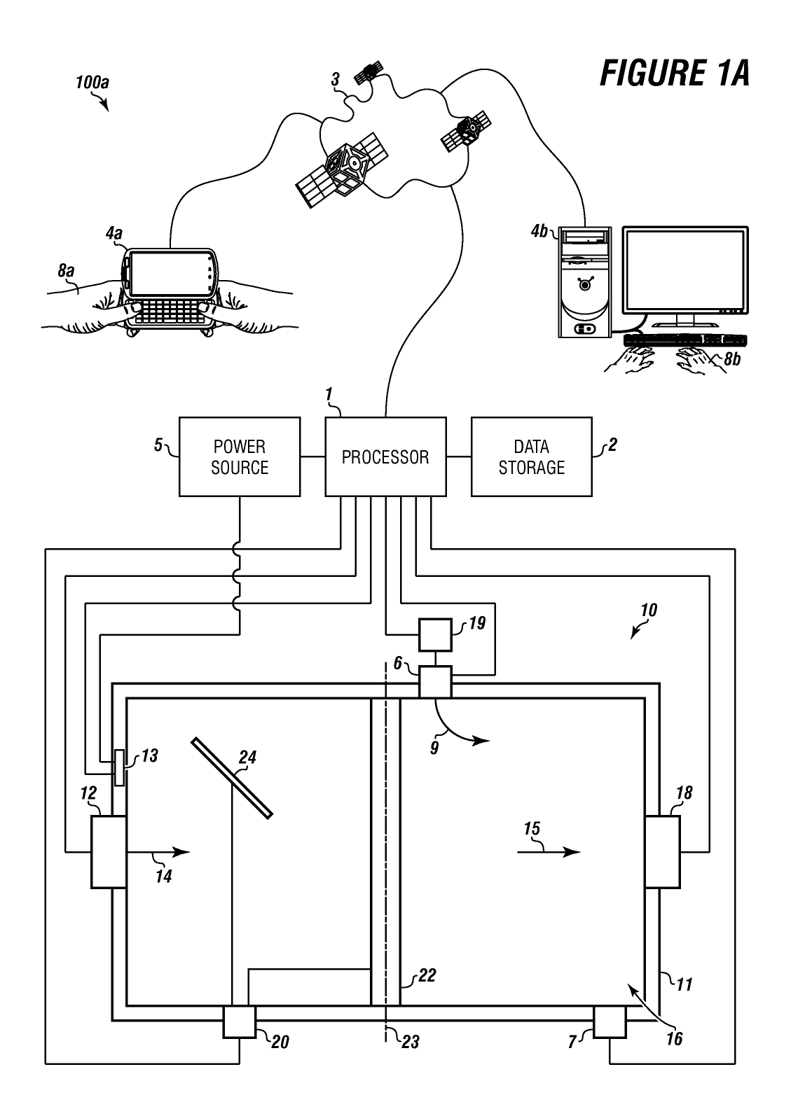

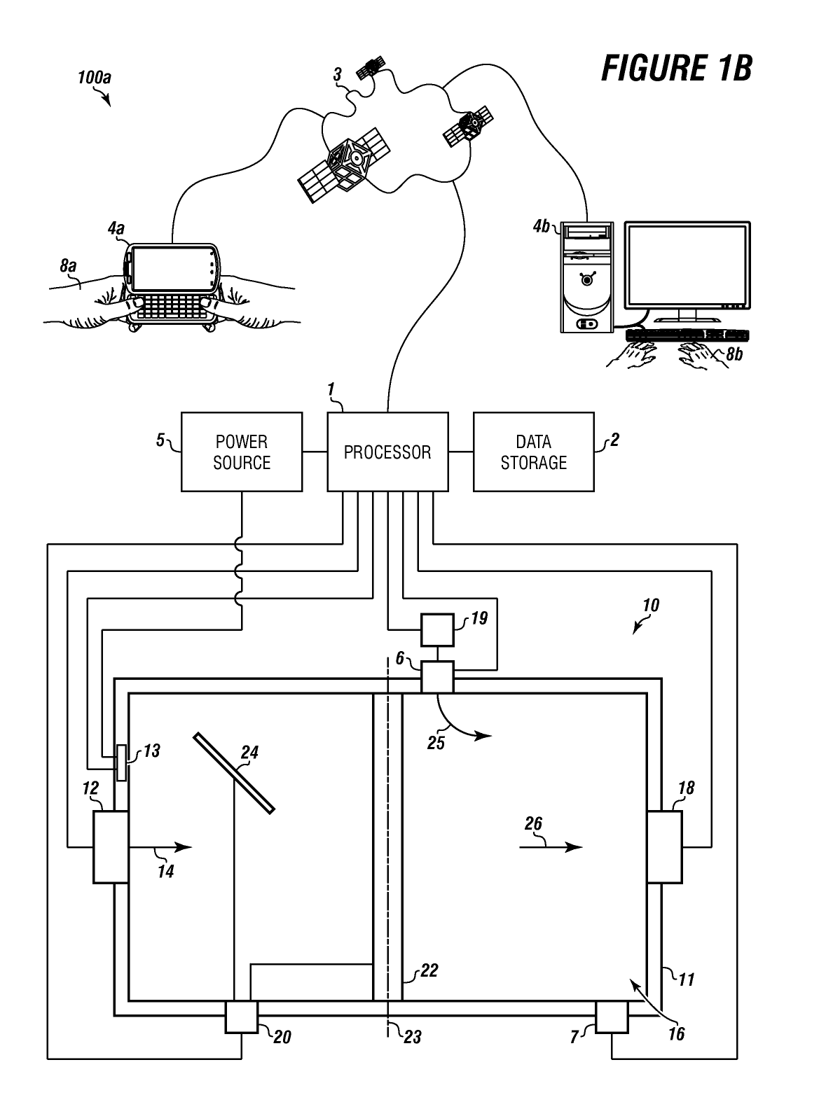

Turning now to the figures, FIG. 1A depicts an embodiment of a system that can be used to implement the method for testing a gas sample, and FIG. 1B depicts the system of FIG. 1A testing a calibration gas sample.

The system 100a can include a processor 1 in communication with a power source 5.

The processor 1 can be in communication with one or more client devices 4a and 4b via a network 3, which can be operated by one or more users 8a and 8b. The network 3 can be a wireless network, a wired network, or combinations thereof.

The processor 1 can be in communication with a data storage 2.

The system 100a can also include an inline component gas analyzer 10 having a sample chamber 16.

The sample chamber 16 can have a means to allow a gas sample 9 or calibration gas sample 25 to flow into the sample chamber 16, such as through an inlet 6. The inlet 6 can be in communication with the processor 1, allowing the processor 1 to control opening, closing, and adjustment of the inlet 6. In one or more embodiments, the inlet 6 can include a valve.

In one or more embodiments, the inline component gas analyzer 10 can include a bullet-proof and water-proof enclosure 11. The bullet-proof and water-proof enclosure 11 can be installed around the inline component gas analyzer 10, and can be sufficiently rigid to sustain a drop of five feet onto concrete without deforming the inline component gas analyzer 10. The bullet-proof and water-proof enclosure 11 can be made of steel or a non-deforming polymer.

The system 100a can include a pump 19 for pumping the gas sample 9 or calibration gas sample 25 through the inlet 6. The pump 19 can be in communication with the processor 1, allowing the processor 1 to control the pump 19.

In one or more embodiments, the pump 19 can be a positive pressure pump or a vacuum pressure pump for introducing the gas sample 9 or calibration gas sample 25 into the sample chamber 16 through the inlet 6.

A filter frame 22 can be disposed in the sample chamber 16. The filter frame 22 can have one or more filters installed therein.

The system 100a can include one or more minors 24 disposed in the sample chamber 16.

The system 100a can include a means for sliding the filter frame, the mirrors, or combinations thereof, such as a motor 20. The motor 20 can be in mechanical communication with the filter frame 22 and the mirrors 24, in electrical communication with the filter frame 22 and the mirrors 24, or combinations thereof for moving the filter frame 22 and the mirrors 24 within the sample chamber 16. For example, the filter frame 22 and mirrors 24 can be moved along a direction that is perpendicular to a centerline 23 of the sample chamber 16.

The motor 20 can be in communication with the processor 1, allowing the processor 1 to control the movement of the filter frame 22 and mirrors 24. In one or more embodiments, the means for sliding the filter frame 22 can be a robot.

The motor 20 can slide the minors 24 into a path of a beam 14. In one or more embodiments, the system 100a can include a combination of mirrors 24 and filters frames 22 with filters arranged into various configurations.

The system 100a can include an electromagnetic beam generator 12, which can be in communication with the processor 1, allowing the processor 1 to control the electromagnetic beam generator 12.

The electromagnetic beam generator 12 can be configured to generate the beam 14 and transmit the beam 14 into the sample chamber 16.

In operation, the beam 14 can pass from the electromagnetic beam generator 12 into the sample chamber 16, impact the one or more minors 24, and pass through the one or more filters in the filter frame 22. When the beam 14 passes through the one or more of the filters in the filter frame 22, a sample wavelength 15 can be formed and can pass through the gas sample 9, or a calibration wavelength 26 can be formed and can pass through the calibration gas sample 25.

After passing through the gas sample 9 or calibration gas sample 25, the sample wavelength 15 or calibration wavelength 26 can be transmitted from the sample chamber 16 into an electromagnetic beam detector 18 of the system 100a. The electromagnetic beam detector 18 can be in communication with the processor 1.

The system 100a can include a means for exhausting the gas sample 9 or calibration sample 25 from the sample chamber 16, such as though an outlet 7 with a valve. The processor 1 can be in communication with the outlet 7 for controlling the outlet 7. The outlet 7 can allow the gas sample 9 or calibration gas sample 25 to exit the sample chamber 16 after being impacted by the beam 14.

The system 100a can include a computer controlled environmental controller 13 for maintaining the inline component gas analyzer 10 in a climate controlled manner. The computer controlled environmental controller 13 can be contained within the bullet-proof and water-proof enclosure 11 and in communication with the processor 1 and the power supply 5, such that the processor 1 can control the computer controlled environmental controller 13.

The computer controlled environmental controller 13 can be a heating and cooling device configured to maintain the inline component gas analyzer 10 at equipment limits for temperature and humidity.

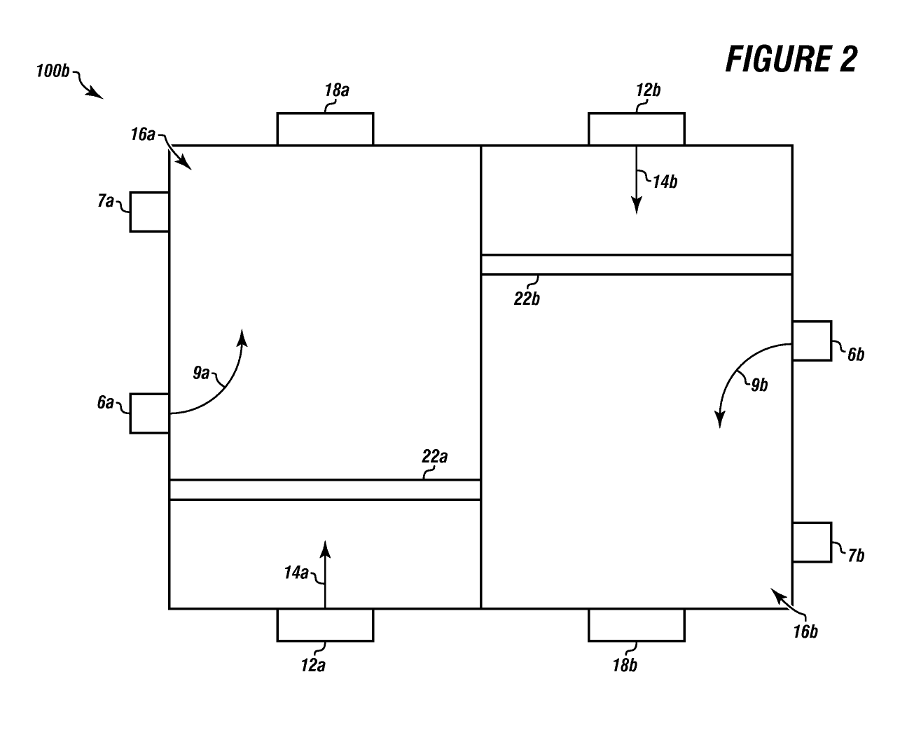

FIG. 2 depicts an embodiment of the system having two sample chambers.

In one or more embodiments, the system 100b can be self-contained, automatic, and can weigh less than 20 pounds. The system 100b can include two sample chambers 16a and 16b.

The system 100b can include two electromagnetic beam generators 12a and 12b, two electromagnetic beam detectors 18a and 18b, and two filter frames 22a and 22b having filters; thereby increasing sensitivity of detection, increasing a quantity of components for detection, or combinations thereof.

The electromagnetic beam generators 12a and 12b can be disposed opposite each other, and the electromagnetic beam detectors 18a and 18b can be disposed opposite each other.

The system 100b can include two inlets 6a and 6b to allow two gas samples 9a and 9b to flow into the sample chambers 16a and 16b.

In one or more embodiments, calcium chloride can be disposed in the sample chambers 16a and 16b to assist in the removal of moisture from the gas samples 9a and 9b.

The system 100b can include two outlets 7a and 7b to exhaust the gas samples 9a and 9b from the sample chambers 16a and 16b.

The electromagnetic beam generators 12a and 12b can generate two beams 14a and 14b to enable for both gas samples 9a and 9b to be simultaneously analyzed.

In operation, the system 100b can be used to simultaneously detect two streams of gas samples 9a and 9b to provide for a faster automated process, or the system 100b can be used to perform duplicate detestation of a single stream of a gas sample to provide for higher accuracy of detection thereof.

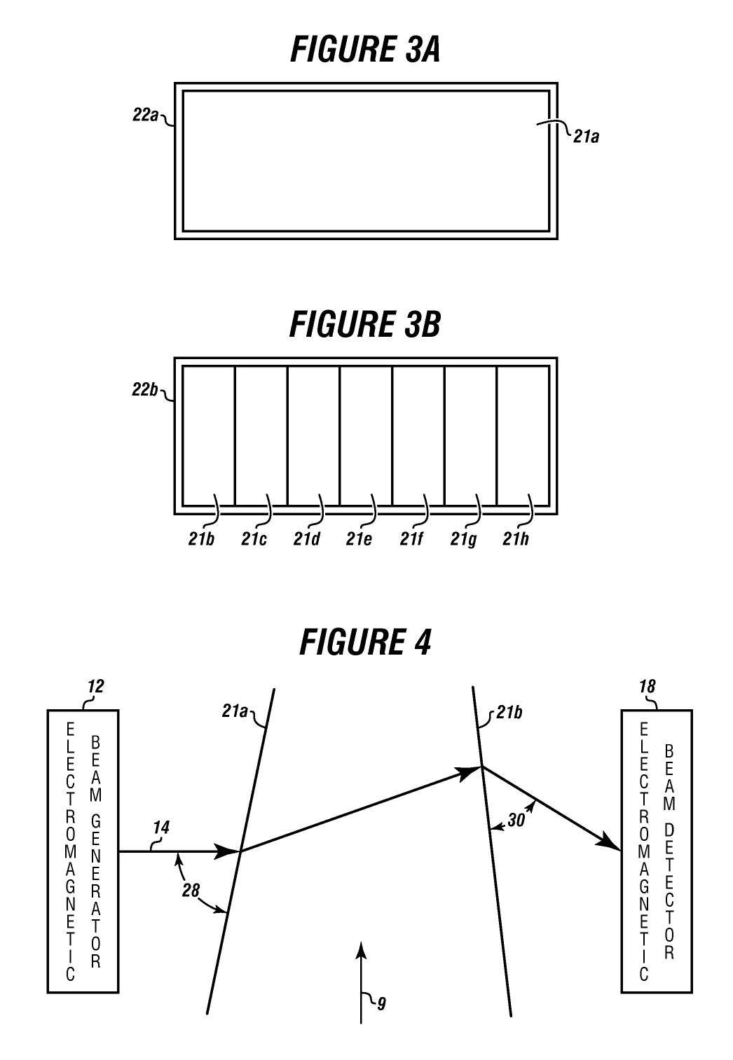

FIG. 3A depicts an embodiment of the filter frame having a single filter and FIG. 3B depicts an embodiment of the filter frame having a plurality of filters.

The filter frame 22a can have a single filter 21a, which can be moved by the means for sliding a filter, such as the motor, robot, or combinations thereof. The means for sliding a filter can slide the filter 21a into the path of the beam produced from the electromagnetic beam generator.

In one or more embodiments, the filter frame 22b can have a plurality of filters 21b, 21c, 21d, 21e, 21f, 21g, and 21h.

The means for sliding a filter can slide the plurality of filters 21b-21h into the path of the beam produced from the electromagnetic beam generator.

In operation, as the beam passes through the filters 21a-21h, each filter 21a-21h can be configured to only allow one of a plurality of the wavelengths that correspond to a hydrocarbon component to pass therethrough. For example, filter 21b can be configured for a wavelength that corresponds to C1, filter 21c can be configured for a wavelength that corresponds to C2, filter 21d can be configured for a wavelength that corresponds to C3, filter 21e can be configured for a wavelength that corresponds to iso-C4, filter 21f can be configured for a wavelength that corresponds to normal-C4, filter 21g can be configured for a wavelength that corresponds to iso-C5, and filter 21c can be configured for a wavelength that corresponds to normal-C5. The hydrocarbon components can also be other components from drilling fluid.

As such, each filter 21a-21h can correspond to a selected wavelength for detection of a molecule, atom, or combinations thereof in the gas sample or calibration gas sample.

FIG. 4 depicts the transmission of the beam through filters.

The electromagnetic beam generator 12 can generate the beam 14, which can transmit through a first filter 21a, through the gas sample 9, through a second filter 21b, and to the electromagnetic beam detector 18.

The beam 14 can pass into the first filter 21a at a first angle of incidence 28, which can range from about 60 degrees to about 90 degrees.

The beam 14 can pass out of the second filter 21b at a second angle of incidence 30, which can range from about 60 degrees to about 90 degrees.

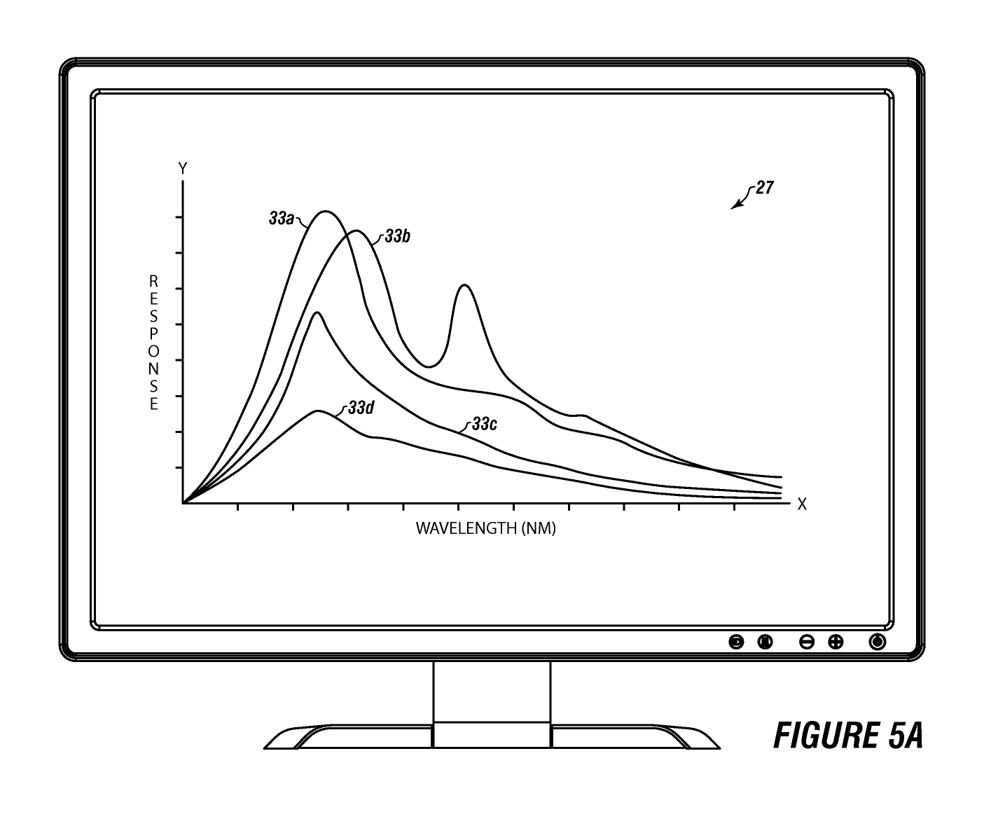

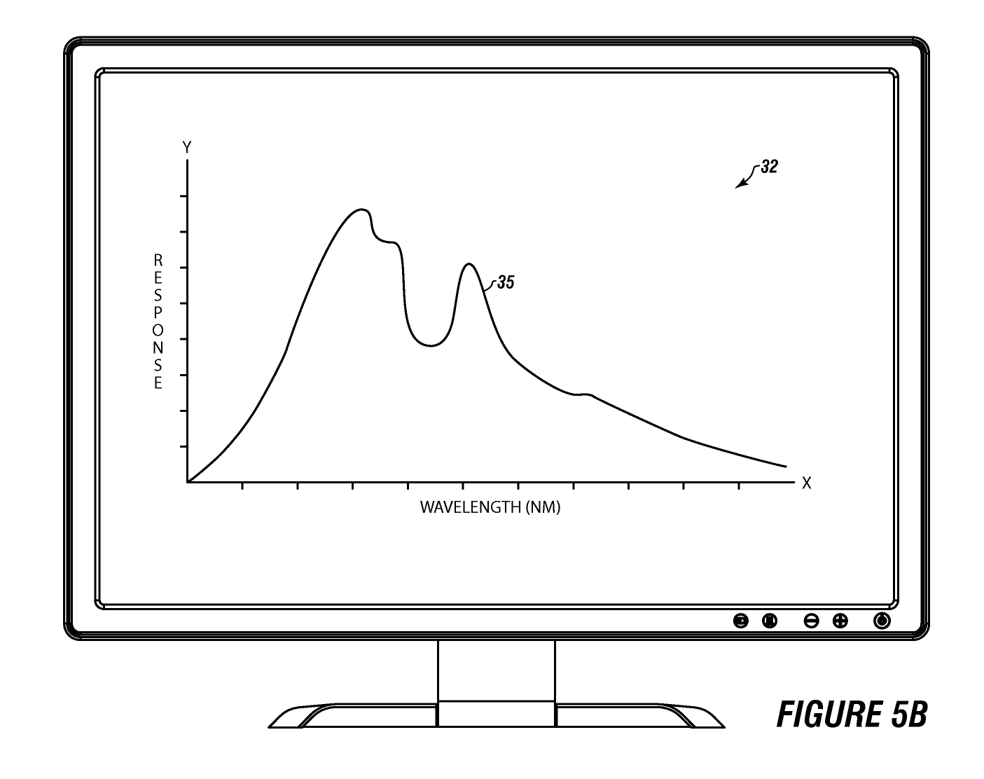

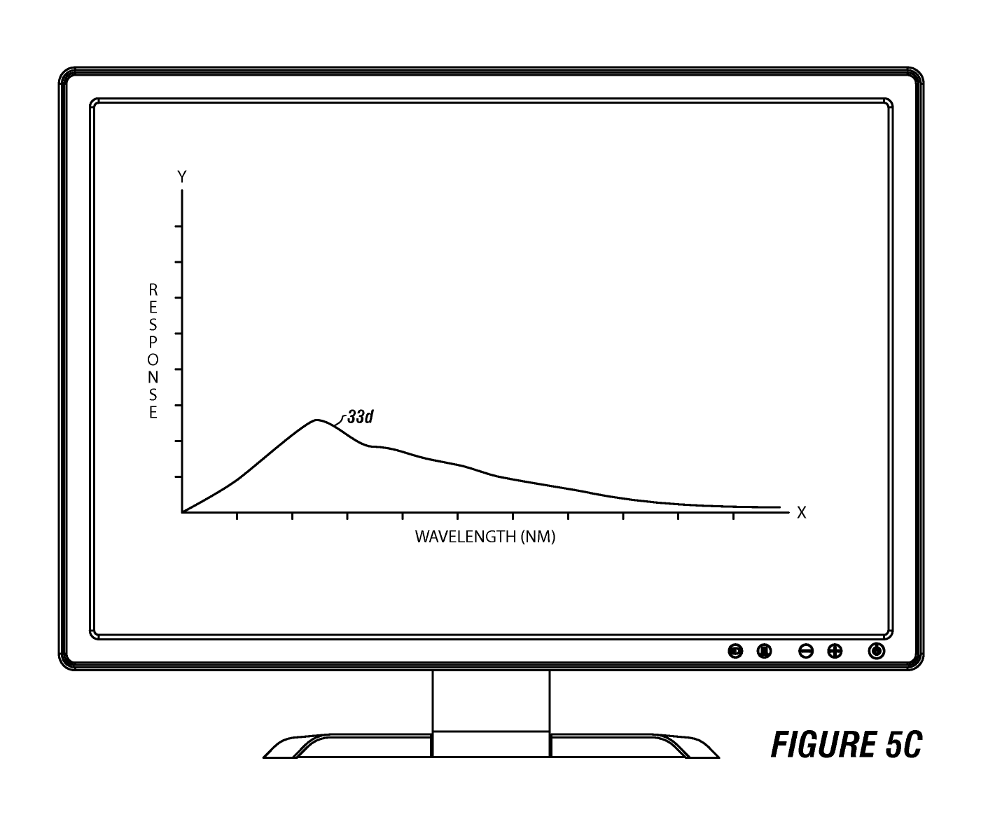

FIG. 5A depicts an embodiment of a calibration radiation matrix.

The calibration radiation matrix 27 can be formed by response curves 33a, 33b, 33c, and 33d, such as response curves for C1, C2, C3, and C4, or the like.

The x-axis can plot wavelength and the y-axis can plot response. The wavelength of the x-axis can be measured in nanometers (nm) or the like.

FIG. 5B depicts an embodiment of an overall spectral data matrix.

The overall spectral data matrix 32 can be formed by a composite response curve 35.

The overall spectral data matrix 32 can be a multicomponent matrix of the gas sample showing, such as for components C1, C2, C3, and C4, or the like.

The x-axis can plot wavelength and the y-axis can plot response.

FIG. 5C depicts a response curve from a single component of a gas sample.

The response curve 33d can be from a single component of a gas sample, such as C4, from the calibration radiation matrix.

The x-axis can plot wavelength and the y-axis can plot response.

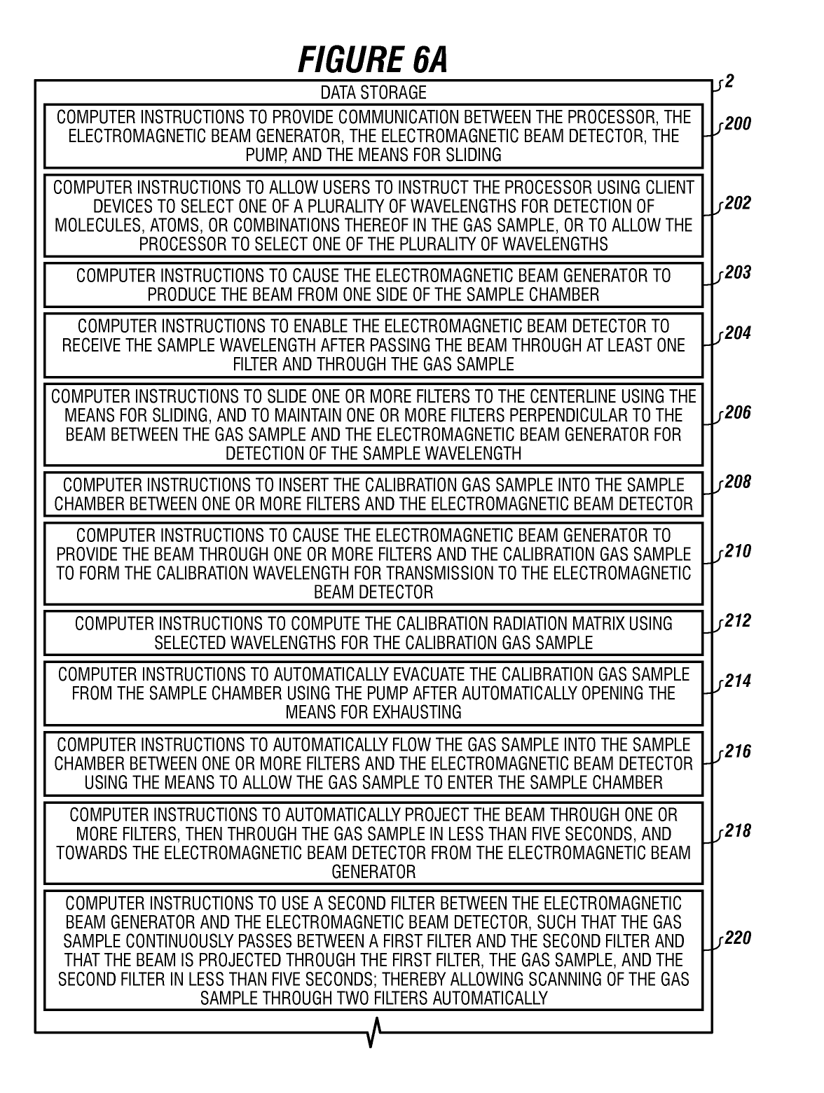

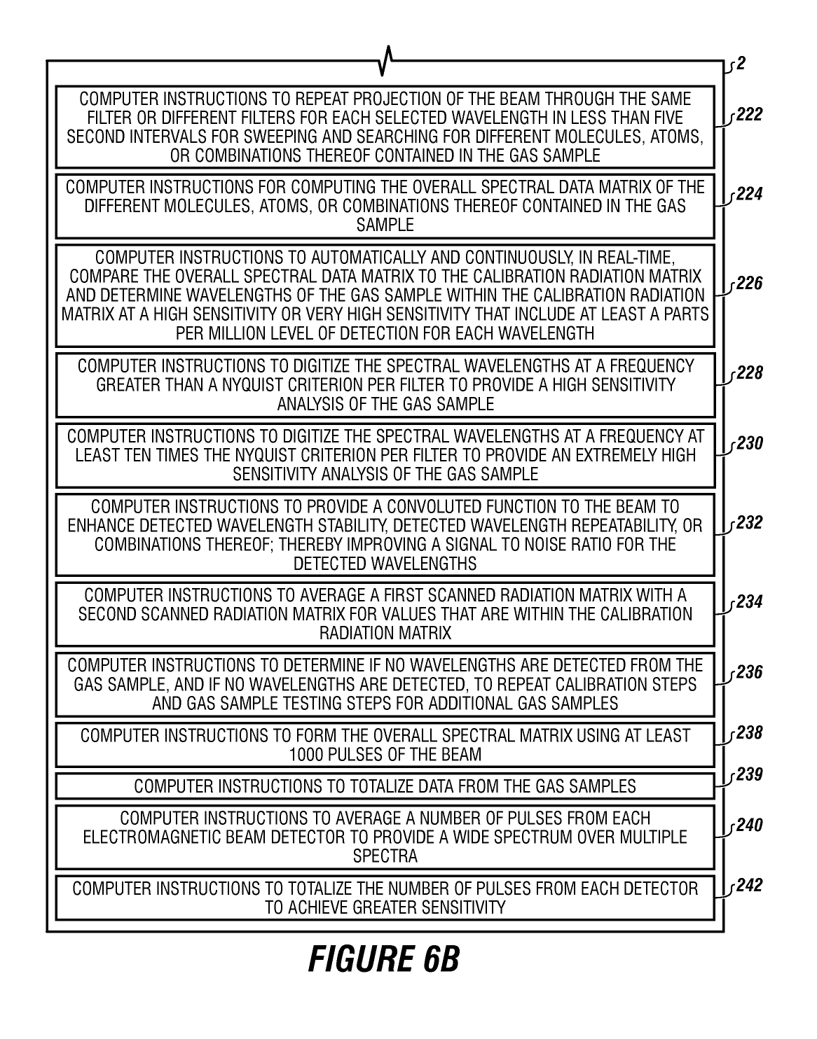

FIGS. 6A and 6B depict the data storage having a plurality of computer instructions for implementing the method according to one or more embodiments.

The data storage 2 can include computer instructions to provide communication between the processor, the electromagnetic beam generator, the electromagnetic beam detector, the pump, and the means for sliding 200. These computer instructions can access telemetry information and other communication information, such as IP address, or identifiers, stored in data storage, for communication devices connected with the electromagnetic beam generator, the electromagnetic beam detector, the pump, and the means for sliding and provide the information to the processor, and the processor can use a communication device, such as those known in the art, connected therewith to and the communication information to communicate with the the electromagnetic beam generator, the electromagnetic beam detector, the pump, and the means for sliding.

The data storage 2 can include computer instructions to allow users to instruct the processor using client devices to select one of a plurality of wavelengths for detection of molecules, atoms, or combinations thereof in the gas sample, or to allow the processor to select one of the plurality of wavelengths 202. The user can enter a desired wavelength, select from a list of wavelengths, or otherwise provide input on a desired wavelength and the computer instructions can tell the processor what wavelength to detect base on the user's input.

The data storage 2 can include computer instructions to cause the electromagnetic beam generator to produce the beam from one side of the sample chamber 203. These computer instructions can allow the user to start the electromagnetic beam generator manually by entering a command, for example by clicking a start icon, or these computer instructions can turn on the generator based on a stored condition, such as time and date, a delay, or the like.

The data storage 2 can include computer instructions to enable the electromagnetic beam detector to receive the sample wavelength after passing the beam through at least one filter and through the gas sample 204.

The data storage 2 can include computer instructions to slide one or more filters to the centerline using the means for sliding, and to maintain one or more filters perpendicular to the beam between the gas sample and the electromagnetic beam generator for detection of the sample wavelength 206.

The data storage 2 can include computer instructions to insert the calibration gas sample into the sample chamber between one or more filters and the electromagnetic beam detector 208.

The data storage 2 can include computer instructions to cause the electromagnetic beam generator to provide the beam through one or more filters and the calibration gas sample to form the calibration wavelength for transmission to the electromagnetic beam detector 210.

The data storage 2 can include computer instructions to compute the calibration radiation matrix using selected wavelengths for the calibration gas sample 212.

The data storage 2 can include computer instructions to automatically evacuate the calibration gas sample from the sample chamber using the pump after automatically opening the means for exhausting 214.

The data storage 2 can include computer instructions to automatically flow the gas sample into the sample chamber between one or more filters and the electromagnetic beam detector using the means to allow the gas sample to enter the sample chamber 216.

The data storage 2 can include computer instructions to automatically project the beam through one or more filters, then through the gas sample in less than five seconds, and towards the electromagnetic beam detector from the electromagnetic beam generator 218.

The data storage 2 can include computer instructions to use a second filter between the electromagnetic beam generator and the electromagnetic beam detector, such that the gas sample continuously passes between a first filter and the second filter and that the beam is projected through the first filter, the gas sample, and the second filter in less than five seconds; thereby allowing scanning of the gas sample through two filters automatically 220.

The data storage 2 can include computer instructions to repeat projection of the beam through the same filter or different filters for each selected wavelength in less than five second intervals for sweeping and searching for different molecules, atoms, or combinations thereof contained in the gas sample 222.

The data storage 2 can include computer instructions for computing the overall spectral data matrix of the different molecules, atoms, or combinations thereof contained in the gas sample 224.

The data storage 2 can include computer instructions to automatically and continuously, in real-time, compare the overall spectral data matrix to the calibration radiation matrix and determine wavelengths of the gas sample within the calibration radiation matrix at a high sensitivity or very high sensitivity that include at least a parts per million level of detection for each wavelength 226.

The data storage 2 can include computer instructions to digitize the spectral wavelengths at a frequency greater than a Nyquist criterion per filter to provide a high sensitivity analysis of the gas sample 228.

The data storage 2 can include computer instructions to digitize the spectral wavelengths at a frequency at least ten times the Nyquist criterion per filter to provide an extremely high sensitivity analysis of the gas sample 230.

The data storage 2 can include computer instructions to provide a convoluted function to the beam to enhance detected wavelength stability, detected wavelength repeatability, or combinations thereof; thereby improving a signal to noise ratio for the detected wavelengths 232.

The convoluted function can be an algorithm that pulses electromagnetic radiation to avoid occurrence of noise; thereby increasing a signal to noise ratio.

The data storage 2 can include computer instructions to average a first scanned radiation matrix with a second scanned radiation matrix for values that are within the calibration radiation matrix 234.

The data storage 2 can include computer instructions to determine if no wavelengths are detected from the gas sample and if no wavelengths are detected, to repeat calibration steps and gas sample testing steps for additional gas samples 236.

The data storage 2 can include computer instructions to form the overall spectral matrix using at least 1000 pulses of the beam 238.

The data storage 2 can include computer instructions to totalize data from the gas samples 239.

The data storage 2 can include computer instructions to average a number of pulses from each electromagnetic beam detector to provide a wide spectrum over multiple spectra 240.

The data storage 2 can include computer instructions to totalize the number of pulses from each detector to achieve greater sensitivity 242.

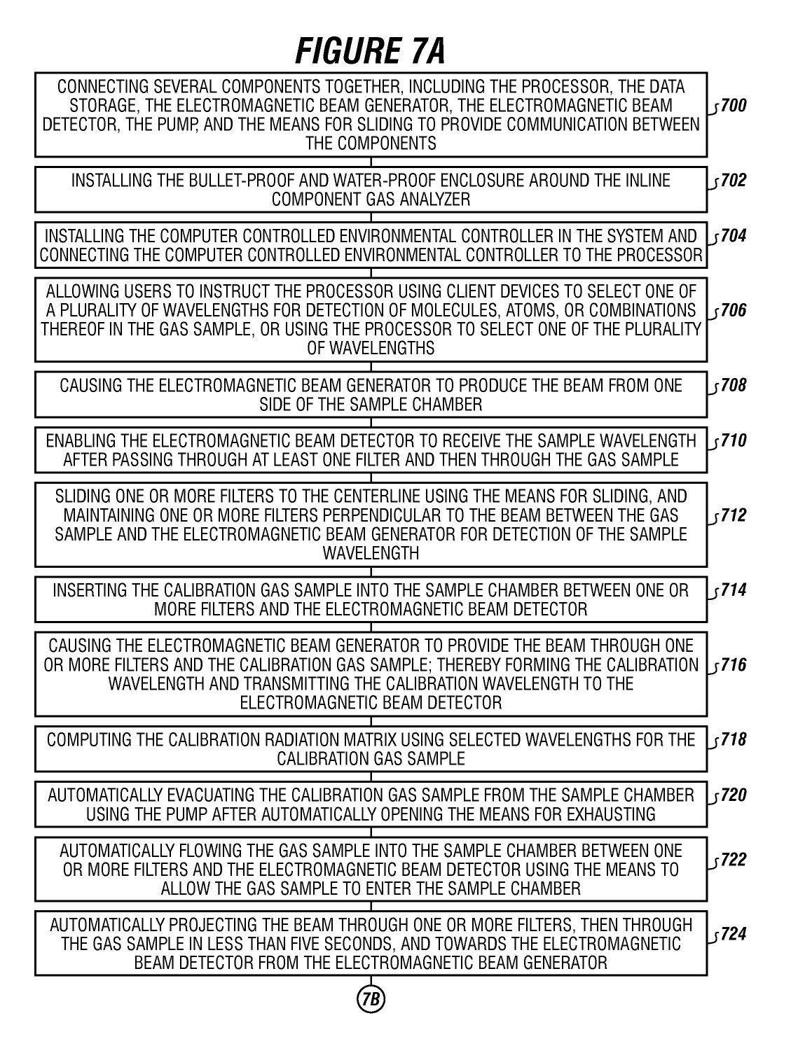

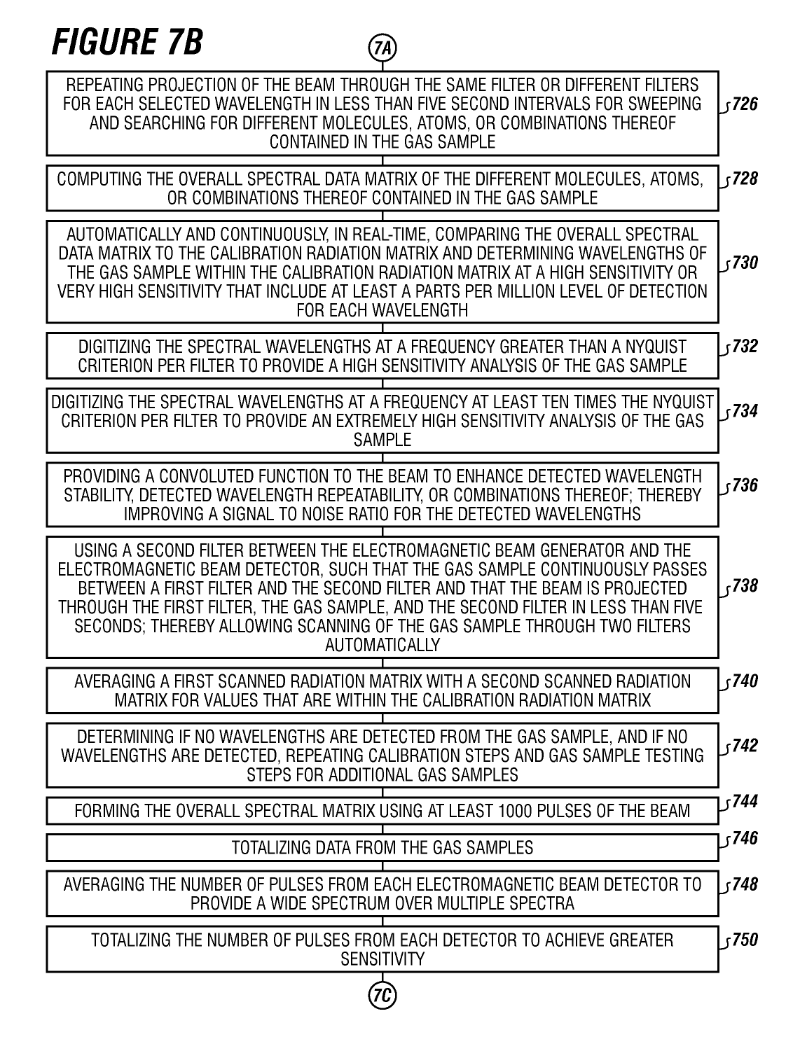

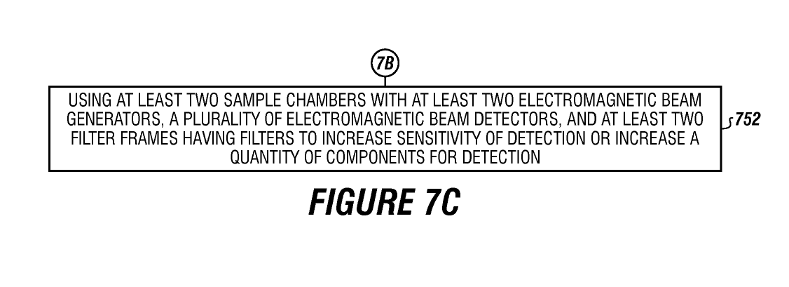

FIGS. 7A-7C depict an embodiment of the method.

The method can include connecting several components together, including the processor, the data storage, the electromagnetic beam generator, the electromagnetic beam detector, the pump, and the means for sliding to provide communication between the components, as illustrated by box 700.

The method can include installing the bullet-proof and water-proof enclosure around the inline component gas analyzer, as illustrated by box 702.

The method can include installing the computer controlled environmental controller in the system and connecting the computer controlled environmental controller to the processor, as illustrated by box 704.

The method can include allowing users to instruct the processor using client devices to select one of a plurality of wavelengths for detection of molecules, atoms, or combinations thereof in the gas sample, or using the processor to select one of the plurality of wavelengths, as illustrated by box 706.

The method can include causing the electromagnetic beam generator to produce the beam from one side of the sample chamber, as illustrated by box 708.

The method can include enabling the electromagnetic beam detector to receive the sample wavelength after passing through at least one filter and then through the gas sample, as illustrated by box 710.

The method can include sliding one or more filters to the centerline using the means for sliding, and maintaining one or more filters perpendicular to the beam between the gas sample and the electromagnetic beam generator for detection of the sample wavelength, as illustrated by box 712.

The method can include inserting the calibration gas sample into the sample chamber between one or more filters and the electromagnetic beam detector, as illustrated by box 714.

The method can include causing the electromagnetic beam generator to provide the beam through one or more filters and the calibration gas sample; thereby forming the calibration wavelength and transmitting the calibration wavelength to the electromagnetic beam detector, as illustrated by box 716.

The method can include computing the calibration radiation matrix using selected wavelengths for the calibration gas sample, as illustrated by box 718.

The method can include automatically evacuating the calibration gas sample from the sample chamber using the pump after automatically opening the means for exhausting, as illustrated by box 720.

The method can include automatically flowing the gas sample into the sample chamber between one or more filters and the electromagnetic beam detector using the means to allow the gas sample to enter the sample chamber, as illustrated by box 722.

The method can include automatically projecting the beam through one or more filters, then through the gas sample in less than five seconds, and towards the electromagnetic beam detector from the electromagnetic beam generator, as illustrated by box 724.

The method can include repeating projection of the beam through the same filter or different filters for each selected wavelength in less than five second intervals for sweeping and searching for different molecules, atoms, or combinations thereof contained in the gas sample, as illustrated by box 726.

The method can include computing the overall spectral data matrix of the different molecules, atoms, or combinations thereof contained in the gas sample, as illustrated by box 728.

The method can include automatically and continuously, in real-time, comparing the overall spectral data matrix to the calibration radiation matrix and determining wavelengths of the gas sample within the calibration radiation matrix at a high sensitivity or very high sensitivity that include at least a parts per million level of detection for each wavelength, as illustrated by box 730.

The method can include digitizing the spectral wavelengths at a frequency greater than a Nyquist criterion per filter to provide a high sensitivity analysis of the gas sample, as illustrated by box 732.

The method can include digitizing the spectral wavelengths at a frequency at least ten times the Nyquist criterion per filter to provide an extremely high sensitivity analysis of the gas sample, as illustrated by box 734.

The method can include providing a convoluted function to the beam to enhance detected wavelength stability, detected wavelength repeatability, or combinations thereof; thereby improving a signal to noise ratio for the detected wavelengths, as illustrated by box 736.

The method can include using a second filter between the electromagnetic beam generator and the electromagnetic beam detector, such that the gas sample continuously passes between a first filter and the second filter and that the beam is projected through the first filter, the gas sample, and the second filter in less than five seconds; thereby allowing scanning of the gas sample through two filters automatically, as illustrated by box 738.

The method can include averaging a first scanned radiation matrix with a second scanned radiation matrix for values that are within the calibration radiation matrix, as illustrated by box 740.

The method can include determining if no wavelengths are detected from the gas sample, and if no wavelengths are detected, repeating calibration steps and gas sample testing steps for additional gas samples, as illustrated by box 742.

The method can include forming the overall spectral matrix using at least 1000 pulses of the beam, as illustrated by box 744.

The method can include totalizing data from the gas samples, as illustrated by box 746.

The method can include averaging the number of pulses from each electromagnetic beam detector to provide a wide spectrum over multiple spectra, as illustrated by box 748.

The method can include totalizing the number of pulses from each detector to achieve greater sensitivity, as illustrated by box 750.

The method can include using at least two sample chambers with at least two electromagnetic beam generators, a plurality of electromagnetic beam detectors, and at least two filter frames having filters to increase sensitivity of detection or increase a quantity of components for detection, as illustrated by box 752.

While these embodiments have been described with emphasis on the embodiments, it should be understood that within the scope of the appended claims, the embodiments might be practiced other than as specifically described herein.

What is claimed is:

1. A computer implemented, real-time, continuous, electromagnetic, spectroscopic, high sensitivity, digital method for analyzing a gas sample using an inline component gas analyzer, the method comprising:
a. connecting a processor in communication with a data storage to a network for communication with a plurality of client devices;
b. connecting the processor with a power source, wherein the processor uses computer instructions in the data storage to communicate with and control: an electromagnetic beam generator, an electromagnetic beam detector, a pump, a means for selectively allowing the gas sample or a calibration gas sample to enter a sample chamber, a means for exhausting the gas sample or the calibration gas sample from the sample chamber, and a means for sliding, wherein the means for sliding is configured to slide one or more filters, one or more filter frames, one or more minors, or combinations thereof;
c. using computer instructions in the data storage to enable the processor to select one of a plurality of wavelengths for detection of molecules, atoms, or combinations thereof in the gas sample, wherein each of the plurality of the wavelengths corresponds to a hydrocarbon component selected from a group consisting of: C1, C2, C3, iso-C4, normal-C4, iso-C5, normal-C5, or another component from a drilling fluid;
d. using computer instructions in the data storage to slide a first filter of the one or more filters to a centerline of the sample chamber using the means for sliding, and to maintain the first filter perpendicular to the beam between the gas sample and the electromagnetic beam generator for detection of a first sample wavelength;
e. using computer instructions in the data storage to cause the electromagnetic beam generator to produce a beam from one side of the sample chamber;
f. using computer instructions in the data storage to enable the electromagnetic beam detector to receive the beam after the beam passes through the one or more filters and the gas sample, wherein each filter corresponds to a selected wavelength for detection of the molecules, atoms, or combinations thereof in the gas sample;
g. using computer instructions in the data storage to insert the calibration gas sample into the sample chamber between the first filter and the electromagnetic beam detector;
h. using computer instructions in the data storage to cause the electromagnetic beam generator to provide the beam through the first filter and then through the calibration gas sample to form a calibration wavelength that is received by the electromagnetic beam detector;
i. using computer instructions in the data storage to compute a calibration radiation matrix using the sample wavelength for the calibration gas sample;
j. using computer instructions in the data storage to automatically evacuate the calibration gas sample from the sample chamber using the pump after automatically opening the means for exhausting the gas sample;
k. using computer instructions in the data storage to automatically flow the gas sample into the sample chamber between the first filter and the electromagnetic beam detector using the means to allow the gas sample to enter the sample chamber;
l. using computer instructions in the data storage to automatically project the beam through the first filter, then through the gas sample in less than five seconds, and towards the electromagnetic beam detector;
m. using computer instructions in the data storage to repeat projection of the beam through the first filter or a different filter for all selected wavelengths in less than five second intervals; thereby sweeping and searching for different molecules, atoms, or combinations thereof contained in the gas sample;
n. using computer instructions in the data storage to compute an overall spectral data matrix of the different molecules, atoms, or combinations thereof contained in the gas sample; and
o. using computer instructions in the data storage to automatically and continuously compare the overall spectral data matrix to the calibration radiation matrix in real-time to determine wavelengths of the gas sample within the calibration radiation matrix having a high sensitivity that includes at least a parts per million level of detection for each wavelength.

2. The method of claim 1, wherein the one or more filter frames comprises a plurality of filters connected in parallel.

3. The method of claim 2, further comprising using computer instructions in the data storage to digitize spectral wavelengths at a frequency greater than a Nyquist criterion for each filter of the plurality of filters, thereby providing a high sensitivity analysis of the gas sample.

4. The method of claim 3, further comprising using computer instructions in the data storage to digitize the spectral wavelengths at a frequency at least ten times the Nyquist criterion for each filter of the plurality of filters, thereby providing an extremely high sensitivity analysis of the gas sample.

5. The method of claim 1, further comprising using computer instructions in the data storage to provide a convoluted function to the beam to enhance detected wavelength stability, detected wavelength repeatability, or combinations thereof, thereby improving a signal to noise ratio for detected wavelengths.

6. The method of claim 1, further comprising installing a bullet-proof and water-proof enclosure around the inline component gas analyzer configured to survive a drop of five feet onto concrete without deforming.

7. The method of claim 6, further comprising installing a computer controlled environmental controller in the bullet-proof and water-proof enclosure, connecting the computer controlled environmental controller to the processor, and maintaining the inline component gas analyzer at equipment limits using the computer controlled environmental controller.

8. The method of claim 1, further comprising using computer instructions in the data storage to move a second filter between the electromagnetic beam generator and the electromagnetic beam detector, wherein the gas sample is continuously passed between the first filter and the second filter, wherein the beam is projected through the first filter, through the gas sample, and through the second filter in less than five seconds, thereby allowing scanning of the gas sample through two filters automatically.

9. The method of claim 8, wherein the first filter is at a first angle of incidence to the beam that is greater than 60 degrees and less than 90 degrees.

10. The method of claim 8, wherein the second filter is at a second angle of incidence to the beam that is greater than 60 degrees and less than 90 degrees.

11. The method of claim 1, further comprising using computer instructions in the data storage to average a first scanned radiation matrix with a second scanned radiation matrix for values that are within the calibration radiation matrix.

12. The method of claim 1, further comprising using computer instructions in the data storage to determine if no wavelengths are detected from the gas sample, and if no wavelengths are detected to repeat calibration steps and gas sample testing steps.

13. The method of claim 1, further comprising using computer instructions in the data storage to require that the overall spectral data matrix is formed using at least 1000 pulses of the beam.

14. The method of claim 1, wherein the inline component gas analyzer further comprises:
a. a second sample chamber;
b. a second electromagnetic beam generator;
c. a second electromagnetic beam detector; and
d. a second filter frame moveably disposed within the second sample chamber for receiving a second beam.

15. The method of claim 14, further comprising using computer instructions in the data storage to average a number of pulses from each electromagnetic beam detector to provide a wide spectrum over multiple spectra.

16. The method of claim 15, further comprising using computer instructions in the data storage to totalize the number of pulses from each electromagnetic beam detector to achieve greater sensitivity.
